(19) United States
(12) Patent Application Publication
(10) Pub. No.: US 2026/0200913 A1
(43) Pub. Date: Jul. 16, 2026

(54) ATROPISOMERS OF MAT2A INHIBITORS

(71) Applicant: IDEAYA Biosciences, Inc., South San Francisco, CA (US)

(72) Inventor: Xin LINGHU, South San Francisco, CA (US)

(21) Appl. No.: 19/134,068

(22) PCT Filed: Nov. 29, 2023
(86) PCT No.: PCT/US23/81672

§ 371 Date: May 29, 2025

Related U.S. Application Data
(60) Provisional application No. 63/428,838, filed on Nov. 30, 2022.

(51) Int. Cl. C07D 471/04; A61K 31/519; A61P 35/00

(52) U.S. Cl. CPC C07D 471/04; A61K 31/519; A61P 35/00

(57) ABSTRACT

Disclosed herein are atropisomers of Formula (I) that are methionine adenosyl-transferase 2A (MAT2A) inhibitors. Also disclosed are methods of isolating the atropisomers, pharmaceutical compositions comprising such compounds/compositions, and methods of treating diseases treatable by inhibition of MAT2A such as cancer, including, for example, cancers characterized by reduced or absence of methylthioadenosine phosphorylase (MTAP) activity.

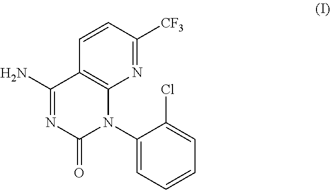 [SMILES: Nc1nc(=O)n(-c2ccccc2Cl)c2nc(C(F)(F)F)ccc12]

### CROSS-REFERENCES TO RELATED APPLICATIONS

This application claims the benefit of priority under 35 U.S.C § 119(e) to U.S. Provisional Application Ser. No. 63/428,838 filed Nov. 30, 2022, the disclosure of which is incorporated herein by reference in its entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Cancer is a leading cause of death throughout the world. A limitation of prevailing therapeutic approaches, e.g. chemotherapy and immunotherapy is that their cytotoxic effects are not restricted to cancer cells and adverse side effects can occur within normal tissues. Consequently, novel strategies are needed to better target cancer cells.

Synthetic lethality arises when a combination of deficiencies in the expression of two or more genes leads to cell death, whereas a deficiency in only one of these genes does not. The concept of synthetic lethality originates from studies in drosophila model systems in which a combination of mutations in two or more separate genes leads to cell death (in contrast to viability, which occurs when only one of the genes is mutated or deleted). More recently, a multitude of studies have explored maladaptive genetic changes in cancer cells that render them vulnerable to synthetic-lethality approaches. These tumor-specific genetic defects lead to the use of targeted agents that induce the death of tumor cells while sparing normal cells.

Methionine adenosyltransferase 2A (MAT2A) is an enzyme that utilizes methionine (Met) and adenosine triphosphate (ATP) to generate s-adenosyl methionine (SAM). SAM is a primary methyl donor in cells used to methylate several substrates including DNA, RNA and proteins. One methylase that utilizes SAM as a methyl donor, is protein arginine N-methyltransferase 5 (PRMT5). While SAM is required for PRMT5 activity, PRMT5 is competitively inhibited by 5′methylthioadenosine (MTA). Since MTA is part of the methionine salvage pathway, cellular MTA levels stay low in a process initiated by methylthioadenosine phosphorylase (MTAP).

MTAP is in a locus on chromosome 9 that is often deleted in cells of patients with cancers from several tissues of origin including central nervous system, pancreas, esophageal, bladder and lung (cBioPortal database). Loss of MTAP results in the accumulation of MTA making MTAP-deleted cells more dependent on SAM production, and thus MAT2A activity, compared to cells that express MTAP. In an shRNA cell-line screen across approximately 400 cancer cell lines, MAT2A knockdown resulted in the loss of viability in a larger percentage of MTAP-deleted cells compare to MTAP WT cells (see McDonald et. al. 2017 Cell 170, 577-592). Furthermore, inducible knockdown of MAT2A protein decreased tumor growth in vivo (see Marjon et. al., 2016 Cell Reports 15(3), 574-587). These results indicate that MAT2A inhibitors may provide a novel therapy for cancer patients including those with MTAP-deleted tumors.

MAT2A inhibitors have been described in, for example, WO2020/123395 filed by Ideaya Biosciences.

Atropisomerism is a form of chirality that arises from hindered rotation around an axis that renders rotational isomers seperatable chemical entities. Many biologically active small molecules possess little hindrance to rotation and exist as a rapidly interconverting mixture of atropisomers, yet bind to their respective biological targets in an atropisomer specific manner. This dynamic nature of atropisomerism can cause serious complications in drug development, as atropisomers can display drastically different pharmacological profiles. This often results in confounding effects caused by the non-target relevant atropisomer, particularly when a compound possesses an intermediate stability, and can racemize over the length of the experiment.

Accordingly, identification and isolation of atropisomers and methods for their preparation and evaluation can provide improved MAT2A inhibitors with enhanced therapeutic properties. The present invention addresses these needs and provides related advantages as well.

### SUMMARY

Disclosed herein are atropisomers of Formula (I):

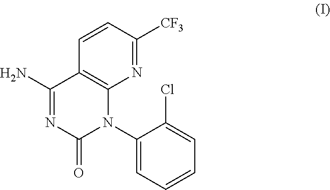 [SMILES: Nc1nc(=O)n(-c2ccccc2Cl)c2nc(C(F)(F)F)ccc12]

that are methionine adenosyltransferase 2A (MAT2A) inhibitors.

In a first aspect provided herein is an atropisomer of Formula (Ia)

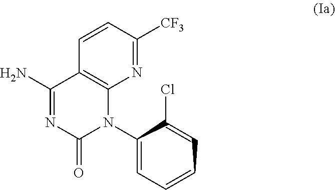

or a pharmaceutically acceptable salt thereof. In some embodiments, provided herein is an atropisomer of Formula (Ib)

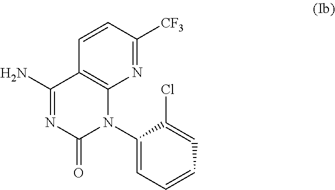

or a pharmaceutically acceptable salt thereof.

In a second aspect, provided herein are compositions comprising an atropisomer of Formula (Ia)

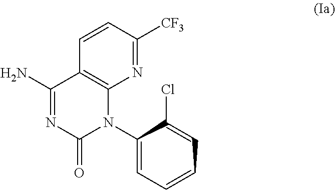

or a pharmaceutically acceptable salt thereof. In some embodiments, provided herein are compositions comprising an atropisomer of Formula (Ib)

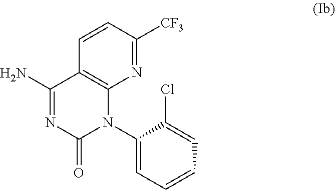

or a pharmaceutically acceptable salt thereof.

In a third aspect, provided is a pharmaceutical composition comprising an atropisomer described herein, or a pharmaceutically acceptable salt thereof, and at least one pharmaceutically acceptable excipient.

In a fourth aspect, provided is a method for treating a disease treatable by inhibition of MAT2A in a patient comprising administering to the patient a therapeutically effective amount of an atropisomer described herein, or a pharmaceutically acceptable salt thereof. In first embodiment of the fourth aspect, the patient is in recognized need of such treatment. In second embodiment of the fourth aspect and first embodiment contained therein, the atropisomer is administered in a pharmaceutical composition. In a third embodiment of the fourth aspect and first and second embodiments contained therein, the disease is cancer.

In a fifth aspect, provided is a method of treating a MTAP null cancer in a patient comprising administering to the patient a therapeutically effective amount of an atropisomer described herein, or a pharmaceutically acceptable salt thereof. In first embodiment of the fifth aspect, the patient is in recognized need of such treatment. In second embodiment of the fifth aspect and first embodiment contained therein, the atropisomer described herein, or a pharmaceutically acceptable salt thereof is administered in a pharmaceutical composition.

In a sith aspect, provided is a method for inhibiting the synthesis of S-adenosyl methionine (SAM) from methionine and ATP by MAT2A in a cell comprising contacting the cell with an effective amount of an atropisomer described herein, or a pharmaceutically acceptable salt thereof.

In a seventh aspect, provided is a method for treating a cancer in a patient, wherein the cancer is characterized by a reduction or absence of methylthioadenosine phosphorylase (MTAP) gene expression, the absence of the MTAP gene, or reduced function of MTAP protein, comprising administering to the subject a therapeutically effective amount of an atropisomer described herein, or a pharmaceutically acceptable salt thereof optionally in a pharmaceutical composition.

In an eighth aspect, provided is an atropisomer described herein, or a pharmaceutically acceptable salt thereof for inhibiting the synthesis of S-adenosyl methionine (SAM) from methionine and ATP by MAT2A in a cell.

In an ninth aspect, provided is an atropisomer described herein, or a pharmaceutically acceptable salt thereof for use in the treatment of a disease in a patient, wherein the disease is mediated by the overexpression of MAT2A.

In a tenth aspect, provided is an atropisomer described herein, or a pharmaceutically acceptable salt thereof for use in the treatment a cancer in a patient, wherein the cancer is characterized by a reduction or absence of methylthioadenosine phosphorylase (MTAP) gene expression, the absence of the MTAP gene, or reduced function of MTAP protein.

In an eleventh aspect, provided is a method for treating a cancer in a patient, wherein the cancer is characterized by a reduction or absence of methylthioadenosine phosphorylase (MTAP) gene expression, the absence of the MTAP gene, reduced level of MTAP protein, or reduced function of MTAP protein, comprising administering to the subject a therapeutically effective amount of an atropisomer described herein, or a pharmaceutically acceptable salt thereof optionally in a pharmaceutical composition.

In a twelfth aspect, provided is an atropisomer described herein, or a pharmaceutically acceptable salt thereof for use in the treatment a cancer in a patient, wherein the cancer is characterized by a reduction or absence of methylthioadenosine phosphorylase (MTAP) gene expression, the absence of the MTAP gene, reduced level of MTAP protein, or reduced function of MTAP protein.

In a thirteenth aspect, provided is an atropisomer described herein, or a pharmaceutically acceptable salt thereof for use in the treatment a cancer in a patient, wherein the cancer is characterized by a reduction or absence of methylthioadenosine phosphorylase (MTAP) gene expression, the absence of the MTAP gene, reduced level of MTAP protein, reduced function of MTAP protein, absence of MTAP protein, MTA accumulation, or combination thereof.

### BRIEF DESCRIPTION OF THE DRAWINGS

NOT APPLICABLE

### DETAILED DESCRIPTION

The present disclosure provides separatable atropisomers of a MAT2A inhibitor. A single atropisomer provides a surprising increase in biologic activity as compared to the other atropisomer.

Before the present invention is further described, it is to be understood that the invention is not limited to the particular embodiments set forth herein, and it is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs.

The publications discussed herein are provided solely for their disclosure prior to the filing date of the present application. Further, the dates of publication provided may be different from the actual publication dates, which may need to be independently confirmed.

### Definitions

Unless otherwise stated, the following terms used in the specification and claims are defined for the purposes of this Application and have the following meaning:

The singular forms “a,” “an,” and “the” as used herein and in the appended claims include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements, or use of a “negative” limitation.

“Pharmaceutically acceptable salts” as used herein is meant to include salts of the active compounds which are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occuring amines and the like, such as arginine, betaine, caffeine, choline. N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogen carbonic, phosphoric, monohydrogen phosphoric, dihydrogen phosphoric, sulfuric, monohydrogen sulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

Certain atropisomers described herein may exist in unsolvated forms as well as solvated forms, including hydrated forms. In general, the solvated forms are equivalent to unsolvated forms and are intended to be encompassed within the scope of the present invention. Certain atropisomers may exist in multiple crystalline or amorphous forms. In general, all physical forms are equivalent for the uses contemplated by the present disclosure and are intended to be within the scope of the present disclosure.

Compounds of Formula (I) or a subembodiment described herein possess atropisomers (isomers based on axial chirality resulting from restricted rotation in the molecule). When a stereochemical depiction is shown, it is meant to refer the compound in which one of the isomers is present in an amount that exceeds the other isomer.

‘Substantially free of” another isomer indicates at least an 80:20 ratio of the two isomers, more preferably 90:10, or 95:5 or more. In some embodiments, one of the isomers will be present in an amount of at least 99%.

The atropisomers described herein may also contain unnatural amounts of isotopes at one or more of the atoms that constitute such compounds. Unnatural amounts of an isotope may be defined as ranging from the amount found in nature to an amount 100% of the atom in question, that differ only in the presence of one or more isotopically enriched atoms. Exemplary isotopes that can be incorporated into compounds of the present invention include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I, respectively. Isotopically-labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, in compounds disclosed herein, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁵F are useful for positron emission tomography (PET) studies to examine substrate receptor occupancy. Isotopically labeled compounds can generally be prepared by following procedures analogous to those disclosed in the Schemes or in the Examples herein, by substituting an isotopically labeled reagent for a non-isotopically labeled reagent.

“Pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass ±10%, preferably ±5%, the recited value and the range is included.

“Disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

“Patient” is generally synonymous with the term “subject” and as used herein includes all mammals including humans. Examples of patients include humans, livestock such as cows, goats, sheep, pigs, and rabbits, and companion animals such as dogs, cats, rabbits, and horses. Preferably, the patient is a human.

“In need of treatment” as used herein refers to a judgment made by a physician or other caregiver that a subject requires or will benefit from treatment. This judgment is made based on a variety of factors that are in the realm of the physician's or caregiver's expertise.

“Administration”, “administer” and the like, as they apply to, for example, a patient, cell, tissue, organ, or biological fluid, refer to contact of, for example, an atropisomer, a pharmaceutically acceptable salt, or a pharmaceutical composition comprising the same to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

“Therapeutically effective amount” as used herein means the amount of an atropisomer described herein and/or a pharmaceutically acceptable salt thereof that, when administered to a patient for treating a disease either alone or as part of a pharmaceutical composition and either in a single dose or as part of a series of doses, is sufficient to affect such treatment for the disease. The “therapeutically effective amount” will vary depending on the compound, the disease and its severity and the age, weight, etc., of the mammal to be treated. The therapeutically effective amount can be ascertained by measuring relevant physiological effects, and it can be adjusted in connection with the dosing regimen and diagnostic analysis of the subject's condition, and the like. By way of example, measurement of the serum level of an atropisomer described herein (or, e.g., a metabolite thereof) at a particular time post-administration may be indicative of whether a therapeutically effective amount has been used.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

“Inhibiting”, “reducing,” or any variation of these terms in relation of MAT2A, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of MAT2A activity compared to its normal activity.

### Compounds of Formula (I)

In some aspects, provided herein are atropisomers of Formula (I)

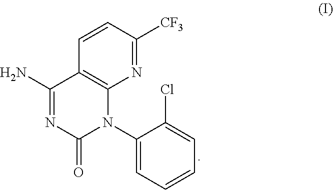 [SMILES: Nc1nc(=O)n(-c2ccccc2Cl)c2nc(C(F)(F)F)ccc12]

The present inventors have identified that the bond connecting the core pyridopyrimidin to the 2-chlorophenyl moiety has sufficient hinderance about the rotational axis that atropisomers can be successfully isolated. The two isolated atropisomers are labeled Formula (Ia) and Formula (Ib) in this disclosure:

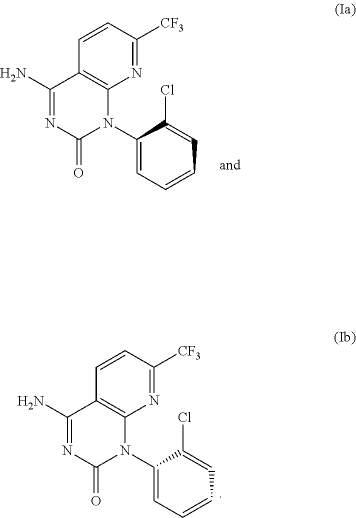

In some embodiments, provided herein is an atropisomer of Formula (Ia)

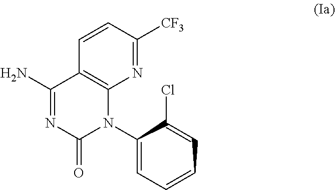

or a pharmaceutically acceptable salt thereof.

In some embodiments, provided herein is an atropisomer of Formula (Ia)

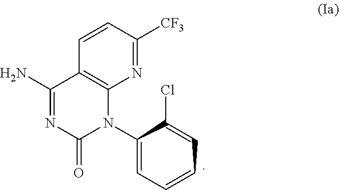

In some embodiments, provided herein is an atropisomer of Formula (Ib)

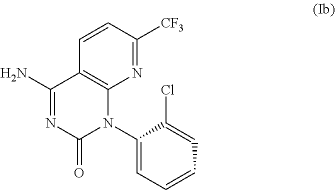

or a pharmaceutically acceptable salt thereof.

In some embodiments, provided herein is an atropisomer of Formula (Ib)

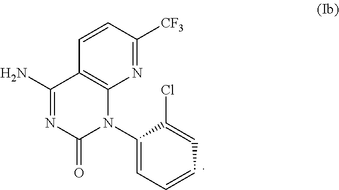

In some embodiments, provided herein are compositions comprising an atropisomer of Formula (Ia)

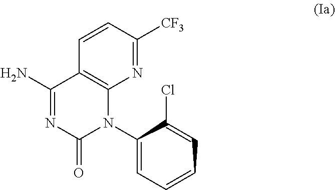

or a pharmaceutically acceptable salt thereof.

In some embodiments, provided herein are compositions comprising an atropisomer of Formula (Ib)

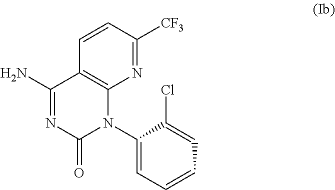

or a pharmaceutically acceptable salt thereof.

In some embodiments the compositions described herein include certain amount of the other atropisomer (i.e., some of Formula Ib in compositions with Formula Ia). Even when the other atropisomer is present in the described compositions, there is an enantiomeric excess of the displayed atropisomer. In some embodiments, the described compositions are substantially free of the other atropisomer.

In some embodiments, the enantiomeric ratio of atropisomers in the described compositions is at least 55:45 of the displayed atropisomer. In some embodiments, the enantiomeric ratio of atropisomers in the described compositions is at least 60:40 of the displayed atropisomer. In some embodiments, the enantiomeric ratio of atropisomers in the described compositions is at least 65:35 of the displayed atropisomer. In some embodiments, the enantiomeric ratio of atropisomers in the described compositions is at least 70:30 of the displayed atropisomer. In some embodiments, the enantiomeric ratio of atropisomers in the described compositions is at least 75:25 of the displayed atropisomer. In some embodiments, the enantiomeric ratio of atropisomers in the described compositions is at least 80:20 of the displayed atropisomer. In some embodiments, the enantiomeric ratio of atropisomers in the described compositions is at least 85:15 of the displayed atropisomer. In some embodiments, the enantiomeric ratio of atropisomers in the described compositions is at least 90:10 of the displayed atropisomer. In some embodiments, the enantiomeric ratio of atropisomers in the described compositions is at least 95:5 of the displayed atropisomer. In some embodiments, the enantiomeric ratio of atropisomers in the described compositions is at least 99:1 of the displayed atropisomer.

In some embodiments, the enantiomeric ratio of atropisomers in the described compositions is at least 75:25, 76:24, 77:23, 78:22, 79:21, 80:20, 81:19, 82:18, 83:17; 84:16, 85:15, 86:14, 87:13, 88:12, 89:11, 90:10, 91:9, 92:8, 93:7, 94:6, 95:5, 96:4, 97:3, 98:2, or 99:1 of the displayed atropisomer.

The enantiomeric ratio can be determined by standard means in the art. As a non-limiting example, Method A in Example 1 of the current application can be used to determine the enantiomeric ratio of atropisomers present in a sample.

Method of making compounds of Formula I have been described previously in, for example, WO2020/123395 filed by Ideaya Biosciences. This reference did not isolate atropisomers nor desecribe how to isolate the currently disclosed atropisomers. Example 1 in the current application describes steps for preparation and isolation of the described atropisomers.

### Utility

Overexpression of the enzyme MAT2A has been demonstrated to mediate certain cancers. In an embodiment, the cancer is neuroblastoma, intestine carcinoma (such as rectum carcinoma, colon carcinoma, familiary adenomatous polyposis carcinoma and hereditary non-polyposis colorectal cancer), esophageal carcinoma, labial carcinoma, larynx carcinoma, hypopharynx carcinoma, tongue carcinoma, salivary gland carcinoma, gastric carcinoma, adenocarcinoma, medullary thyroid carcinoma, papillary thyroid carcinoma, renal carcinoma, kidney parenchym carcinoma, ovarian carcinoma, cervix carcinoma, uterine corpus carcinoma, endometrium carcinoma, chorion carcinoma, pancreatic carcinoma, prostate carcinoma, testis carcinoma, breast carcinoma, urinary carcinoma, melanoma, brain tumors (such as glioblastoma, astrocytoma, meningioma, medulloblastoma and peripheral neuroectodermal tumors), Hodgkin lymphoma, non-Hodgkin lymphoma, Burkitt lymphoma, acute lymphatic leukemia (ALL), chronic lymphatic leukemia (CLL), acute myeloid leukemia (AML), chronic myeloid leukemia (CML), adult T-cell leukemia, hepatocellular carcinoma, gall bladder carcinoma, bronchial carcinoma, small cell lung carcinoma, non-small cell lung carcinoma, multiple myeloma, basalioma, teratoma, retinoblastoma, choroidea melanoma, seminoma, rhabdomyo sarcoma, craniopharyngeoma, osteosarcoma, chondrosarcoma, myosarcoma, liposarcoma, fibrosarcoma, Ewing sarcoma and plasmocytoma.

In another embodiment, the cancer is lung cancer, non-small cell lung (NSLC) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the vagina, carcinoma of the vulva, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. In another embodiment, the cancer is selected from the group consisting of non-small cell lung cancer (squamous and adenocarcinoma), urothelial cancer (bladder and upper urinary tract), esophageal cancer, and gastric cancer.

Methylthioadenosine phosphorylase (MTAP) is an enzyme found in all normal tissues that catalyzes the conversion of methylthioadenosine (MTA) into adenine and 5-methylthio-ribose-1-phosphate. The adenine is salvaged to generate adenosine monophosphate, and the 5-methylthioribose-1-phosphate is converted to methionine and formate. Because of this salvage pathway, MTA can serve as an alternative purine source when de novo purine synthesis is blocked. e.g., with antimetabolites, such as L-alanosine.

Many human and murine malignant cells lack MTAP activity. MTAP deficiency is not only found in tissue culture cells but the deficiency is also present in primary leukemias, gliomas, melanomas, pancreatic cancers, non-small cell lung cancers (NSLC), bladder cancers, astrocytomas, osteosarcomas, head and neck cancers, myxoid chondrosarcomas, ovarian cancers, endometrial cancers, breast cancers, soft tissue sarcomas, non-Hodgkin lymphomas, and mesotheliomas. It has been reported by K. Marjon et al., Cell Reports 15 (2016) 574-587, incorporated herein by reference, that proliferation of cancer cells that are MTAP null is inhibited by knocking down MAT2A expression with shRNA. An MTAP null cancer is a cancer in which the MTAP gene has been deleted or lost or otherwise deactivated or a cancer in which the MTAP protein has a reduced or impaired function.

Accordingly, in an embodiment of the present disclosure there is provided a method for treating an MTAP null cancer in a patient wherein said cancer is characterized by a reduction or absence of MTAP expression or absence of the MTAP gene or reduced function of MTAP protein as compared to cancers where the MTAP gene is present and fully functioning, said method comprising administering to the patient in need thereof a therapeutically effective amount of an atropisomer described herein or a pharmaceutically acceptable salt thereof. In an embodiment of the present disclosure there is provided a method for treating an MTAP null cancer in a patient wherein said cancer is characterized by a reduction or absence of MTAP expression or absence of the MTAP gene, reduced level of MTAP protein, or reduced function of MTAP protein as compared to cancers where the MTAP gene is present and fully functioning, said method comprising administering to the patient in need thereof a therapeutically effective amount of an atropisomer described herein or a pharmaceutically acceptable salt thereof. In another embodiment, provided is a method of treating an MTAP null cancer in a patient comprising administering to the patient in need thereof an effective amount of an atropisomer described herein or a pharmaceutically acceptable salt thereof. In an embodiment, the MTAP null cancer is leukemia, glioma, melanoma, pancreatic, non-small cell lung cancer (NSLC), bladder cancer, astrocytoma, osteosarcoma, head and neck cancer, myxoid chondrosarcoma, ovarian cancer, endometrial cancer, breast cancer, soft tissue sarcoma, non-Hodgkin lymphoma or mesothelioma. In another embodiment, the MTAP null cancer is pancreatic cancer. In yet another embodiment, the MTAP null cancer is bladder cancer, melanoma, brain cancer, lung cancer, pancreatic cancer, breast cancer, esophageal cancer, head and neck cancer, kidney cancer, colon cancer, diffuse large B cell lymphoma (DLBCL), acute lymphoblastic leukemia (ALL) or mantle cell lymphoma (MCL). In yet another embodiment, the MTAP null cancer is gastric cancer. In yet another embodiment, the cancer is colon cancer. In yet another embodiment, the MTAP null cancer is liver cancer. In yet another embodiment, the MTAP null cancer is glioblastoma multiforme (GBM). In yet another embodiment, the MTAP null cancer is bladder cancer. In yet another embodiment, the MTAP null cancer is esophageal cancer. In yet another embodiment, the MTAP null cancer is breast cancer. In yet another embodiment, the MTAP null cancer is NSLCC. In yet another embodiment, the MTAP null cancer is MCL. In yet another embodiment, the MTAP null cancer is DLBCL. In yet another embodiment, the MTAP null cancer is ALL.

In another embodiment, the MTAP null cancer is solid tumor. In another embodiment, the MTAP null cancer is malignant solid tumor.

Genomic analysis of MTAP null cell lines has shown that cell lines that also incorporate a KRAS mutation or a p53 mutation were sensitive to MAT2A inhibition.

Accordingly, also provided is a method for treating a cancer in a patient wherein said cancer is characterized by reduction or absence of MTAP expression or absence of the MTAP gene or reduced function of MTAP protein (i.e., MTAP null) and further characterized by the presence of mutant KRAS and/or mutant p53, said method comprising administering to the patient a therapeutically effective amount of an atropisomer described herein. In one embodiment, the cancer is MTAP null and KRAS mutant. In another embodiment, the cancer is MTAP null and p53 mutant. In yet another embodiment, the cancer is MTAP null, KRAS mutant and p53 mutant.

The term “mutant KRAS” or “KRAS mutation” refers to KRAS protein (or gene encoding said protein) incorporating an activating mutation that alters its normal function. For example, a mutant KRAS protein may incorporate a single amino acid substitution at position 12 or 13. In a particular embodiment, the KRAS mutant incorporates a G12X or G13X substitution, wherein X represents any amino acid change at the indicated position. In a particular embodiment, the substitution is G12V, G12R, G12C or G13D. In another embodiment, the substitution is G13D. By “mutant p53” or “p53 mutation” is meant p53 protein (or gene encoding said protein) incorporating a mutation that inhibits or eliminates its tumor suppressor function. In an embodiment, said p53 mutation is, Y126_splice, K132Q, M133K, R174fs, R175H, R196*, C238S, C242Y, G245S, R248W, R248Q, 1255T, D259V, S261_splice, R267P, R273C, R282W, A159V or R280K. In an embodiment, the foregoing cancer is non-small cell lung cancer (NSLCC), pancreatic cancer, head and neck cancer, gastric cancer, breast cancer, colon cancer or ovarian cancer.

### Assay

The ability of compounds of the disclosure to inhibit MAT2A can be measured as described in Example 3 below.

### Pharmaceutical Composition

The atropisomers described herein, or a pharmaceutically acceptable salt thereof, may be in the form of compositions suitable for administration to a subject. In general, such compositions are pharmaceutical compositions comprising an atropisomer described herein or a pharmaceutically acceptable salt thereof and one or more pharmaceutically acceptable or physiologically acceptable excipients. In certain embodiments, the atropisomer described herein, or a pharmaceutically acceptable salt thereof is present in a therapeutically effective amount. The pharmaceutical compositions may be used in the methods disclosed herein; thus, for example, the pharmaceutical compositions can be administered ex vivo or in vivo to a subject in order to practice the therapeutic methods and uses described herein.

The pharmaceutical compositions can be formulated to be compatible with the intended method or route of administration; exemplary routes of administration are set forth herein. Furthermore, the pharmaceutical compositions may be used in combination with other therapeutically active agents or compounds as described herein in order to treat the diseases, disorders and conditions contemplated by the present disclosure.

The pharmaceutical compositions containing the active ingredient (e.g., an atropisomer described herein, a pharmaceutically acceptable salt thereof) may be in a form suitable for oral use, for example, as tablets, capsules, troches, lozenges, aqueous or oily suspensions, dispersible powders or granules, emulsions, hard or soft capsules, or syrups, solutions, microbeads or elixirs. Pharmaceutical compositions intended for oral use may be prepared according to any method known to the art for the manufacture of pharmaceutical compositions, and such compositions may contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets, capsules, and the like. These excipients may be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration may be uncoated or coated by known techniques to delay disintegration and absorption in the gastrointestinal tract and thereby provide a sustained action. For example, a time-delay material such as glyceryl monostearate or glyceryl di-stearate may be employed. The tablets may also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinyl acetate, methylcellulose, carboxymethylcellulose, protamine sulfate, or lactide and glycolide copolymers, polylactide and glycolide copolymers, or ethylene vinyl acetate copolymers in order to control delivery of an administered composition. For example, the oral agent can be entrapped in microcapsules prepared by coacervation techniques or by interfacial polymerization, by the use of hydroxymethyl cellulose or gelatin-microcapsules or poly (methyl methacrylate) microcapsules, respectively, or in a colloid drug delivery system. Colloidal dispersion systems include macromolecule complexes, nanocapsules, microspheres, microbeads, and lipid-based systems, including oil-in-water emulsions, micelles, mixed micelles, and liposomes. Methods for the preparation of the above-mentioned formulations are known in the art.

Formulations for oral use may also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, (hydroxypropyl)methyl cellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., poly-oxyethylene stearate), or condensation products of ethylene oxide with long chain aliphatic alcohols (e.g., for heptdecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions may also contain one or more preservatives.

Oily suspensions may be formulated by suspending the active ingredient in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions may also be in the form of oil-in-water emulsions. The oily phase may be a vegetable oil, for example olive oil or arachis oil, or a mineral oil, for example, liquid paraffin, or mixtures of these. Suitable emulsifying agents may be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

The pharmaceutical compositions typically comprise a therapeutically effective amount of an atropisomer described herein, or a salt thereof, and one or more pharmaceutically acceptable excipient. Suitable pharmaceutically acceptable excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle may be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it may be stored in sterile vials as a solution, suspension, gel, emulsion, solid, or dehydrated or lyophilized powder. Such formulations may be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments.

Formulations can also include carriers to protect the composition against rapid degradation or elimination from the body, such as a controlled release formulation, including liposomes, hydrogels, prodrugs and microencapsulated delivery systems. For example, a time delay material such as glyceryl monostearate or glyceryl stearate alone, or in combination with a wax, may be employed. Any drug delivery apparatus may be used to deliver an atropisomer described herein, or a salt thereof, including implants (e.g., implantable pumps) and catheter systems, slow injection pumps and devices, all of which are well known to the skilled artisan.

Depot injections, which are generally administered subcutaneously or intramuscularly, may also be utilized to release the atropisomer described herein, or a salt thereof over a defined period of time. Depot injections are usually either solid- or oil-based and generally comprise at least one of the formulation components set forth herein. One of ordinary skill in the art is familiar with possible formulations and uses of depot injections.

The pharmaceutical compositions may be in the form of a sterile injectable aqueous or oleagenous suspension. The suspension may be formulated according to the known art using those suitable dispersing or wetting agents and suspending agents mentioned herein. The sterile injectable preparation may also be a sterile injectable solution or suspension in a non-toxic parenterally-acceptable diluent or solvent, for example, as a solution in 1,3-butane diol. Acceptable diluents, solvents and dispersion media that may be employed include water, Ringer's solution, isotonic sodium chloride solution, Cremophor EL™ (BASF, Parsippany, NJ) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil may be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

Atropisomers described herein, or a salt thereof may also be administered in the form of suppositories for rectal administration or sprays for nasal or inhalation use. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

### Routes of Administration

Atropisomers described herein, or a pharmaceutically acceptable salt thereof, and compositions containing the same may be administered in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation. Depot injections, which are generally administered subcutaneously or intramuscularly, may also be utilized to administer the atropisomers described herein, or a salt thereof over a defined period of time. Particular embodiments of the present invention contemplate oral administration.

### Combination Therapy

The present invention contemplates the use of atropisomers described herein, or a salt thereof in combination with one or more active therapeutic agents (e.g., chemotherapeutic agents) or other prophylactic or therapeutic modalities (e.g., radiation). In such combination therapy, the various active agents frequently have different, complementary mechanisms of action. Such combination therapy may be especially advantageous by allowing a dose reduction of one or more of the agents, thereby reducing or eliminating the adverse effects associated with one or more of the agents. Furthermore, such combination therapy may have a synergistic therapeutic or prophylactic effect on the underlying disease, disorder, or condition.

As used herein. “combination” is meant to include therapies that can be administered separately, for example, formulated separately for separate administration (e.g., as may be provided in a kit), and therapies that can be administered together in a single formulation (i.e., a “co-formulation”).

In certain embodiments, the atropisomers described herein, or a salt thereof are administered or applied sequentially, e.g., where one agent is administered prior to one or more other agents. In other embodiments, the atropisomers described herein, or a salt thereof are administered simultaneously, e.g., where two or more agents are administered at or about the same time; the two or more agents may be present in two or more separate formulations or combined into a single formulation (i.e., a co-formulation). Regardless of whether the two or more agents are administered sequentially or simultaneously, they are considered to be administered in combination for purposes of the present disclosure.

The atropisomers described herein, or a salt thereof may be used in combination with at least one other (active) agent in any manner appropriate under the circumstances. In one embodiment, treatment with the at least one active agent and at least one atropisomer described herein, or a salt thereof is maintained over a period of time. In another embodiment, treatment with the at least one active agent is reduced or discontinued (e.g., when the subject is stable), while treatment with an atropisomer described herein, or a salt thereof is maintained at a constant dosing regimen. In a further embodiment, treatment with the at least one active agent is reduced or discontinued (e.g., when the subject is stable), while treatment with an atropisomer described herein, or a salt thereof is reduced (e.g., lower dose, less frequent dosing or shorter treatment regimen). In yet another embodiment, treatment with the at least one active agent is reduced or discontinued (e.g., when the subject is stable), and treatment with an atropisomer described herein, or a salt thereof is increased (e.g., higher dose, more frequent dosing or longer treatment regimen). In yet another embodiment, treatment with the at least one active agent is maintained and treatment with an atropisomer described herein, or a salt thereof is reduced or discontinued (e.g., lower dose, less frequent dosing or shorter treatment regimen). In yet another embodiment, treatment with the at least one active agent and treatment with an atropisomer described herein, or a salt thereof are reduced or discontinued (e.g., lower dose, less frequent dosing or shorter treatment regimen).

The present disclosure provides methods for treating cancer with an atropisomer described herein, or a salt thereof and at least one additional therapeutic or diagnostic agent.

In some embodiments, an atropisomer described herein, or a salt thereof is administered in combination with at least one additional therapeutic agent, selected from Temozolomide, Pemetrexed, Pegylated liposomal doxorubicin (Doxil), Eribulin (Halaven), Ixabepilone (Ixempra). Protein-bound paclitaxel (Abraxane), Oxaliplatin. Irinotecan, Venatoclax (bcl2 inhibitor), 5-azacytadine, Anti-CD20 therapeutics, such as Rituxan and obinutuzumab, Hormonal agents (anastrozole, exemestand, letrozole, zoladex, lupon eligard), CDK4/6 inhibitors, Palbociclib, Abemaciclib, CPI (Avelumab, Cemiplimab-rwlc, and Bevacizumab.

In certain embodiments, the present disclosure provides methods for treating cancer comprising administration of an atropisomer described herein, or a salt thereof in combination with a signal transduction inhibitor (STI) to achieve additive or synergistic suppression of tumor growth. As used herein, the term “signal transduction inhibitor” refers to an agent that selectively inhibits one or more steps in a signaling pathway. Examples of signal transduction inhibitors (STIs) useful in methods described herein include, but are not limited to: (i) bcr/abl kinase inhibitors (e.g., GLEEVEC); (ii) epidermal growth factor (EGF) receptor inhibitors, including kinase inhibitors and antibodies: (iii) her-2/neu receptor inhibitors (e.g., HERCEPTIN); (iv) inhibitors of Akt family kinases or the Akt pathway (e.g., rapamycin); (v) cell cycle kinase inhibitors (e.g., flavopiridol); and (vi) phosphatidyl inositol kinase inhibitors. Agents involved in immunomodulation can also be used in combination with one or more atropisomers described herein, or a salt thereof for the suppression of tumor growth in cancer patients.

In certain embodiments, the present disclosure provides methods for treating cancer comprising administration of an atropisomer described herein, or a salt thereof in combination with a chemotherapeutic agents. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone: aldophosphamide glycoside: aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide: procarbazine; razoxane; sizofiran: spirogermanium; tenuazonic acid; triaziquone: 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa: taxoids, e.g., paclitaxel and doxetaxel; chlorambucil; gemcitabine; 6-thioguanine; mercaptopurine; methotrexate; platinum and platinum coordination complexes such as cisplatin and carboplatin; vinblastine; etoposide (VP-16); ifosfamide; mitomycin C; mitoxantrone; vincristine; vinorelbine; navelbine; novantrone; teniposide; daunomycin; aminopterin: xeloda; ibandronate: CPT1; topoisomerase inhibitors; difluoromethylomithine (DMFO); retinoic acid; esperamicins; capecitabine; and pharmaceutically acceptable salts, acids or derivatives of any of the above. In a particular embodiment, compounds of the present disclosure are coadministered with a cytostatic compound selected from the group consisting of cisplatin, doxorubicin, taxol, taxotere and mitomycin C. In a particular embodiment, the cytostatic compound is doxorubicin.

Chemotherapeutic agents also include anti-hormonal agents that act to regulate or inhibit hormonal action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, enzalutamide, apalutamide, abiraterone acetate, leuprolide, and goserelin; and pharmaceutically acceptable salts, acids or derivatives of any of the above. In certain embodiments, combination therapy comprises administration of a hormone or related hormonal agent.

The present disclosure also contemplates the use of the atropisomers described herein, or a salt thereof in combination with immune checkpoint inhibitors. The tremendous number of genetic and epigenetic alterations that are characteristic of all cancers provides a diverse set of antigens that the immune system can use to distinguish tumor cells from their normal counterparts. In the case of T cells, the ultimate amplitude (e.g., levels of cytokine production or proliferation) and quality (e.g., the type of immune response generated, such as the pattern of cytokine production) of the response, which is initiated through antigen recognition by the T-cell receptor (TCR), is regulated by a balance between co-stimulatory and inhibitory signals (immune checkpoints). Under normal physiological conditions, immune checkpoints are crucial for the prevention of autoimmunity (i.e., the maintenance of self-tolerance) and also for the protection of tissues from damage when the immune system is responding to pathogenic infection. The expression of immune checkpoint proteins can be dysregulated by tumors as an important immune resistance mechanism. Examples of immune checkpoint inhibitors include but are not limited to CTLA-4, PD-1, PD-L1, BTLA, TIM3, LAG3, OX40, 41BB, VISTA, CD96, TGFβ, CD73, CD39, A2AR, A2BR, IDO1, TDO2, Arginase, B7-H3, B7-H4. Cell-based modulators of anti-cancer immunity are also contemplated. Examples of such modulators include but are not limited to chimeric antigen receptor T-cells, tumor infiltrating T-cells and dendritic-cells.

The present disclosure contemplates the use of atropisomers described herein, or a salt thereof in combination with inhibitors of the aforementioned immune-checkpoint receptors and ligands, for example ipilimumab, abatacept, nivolumab, pembrolizumab, atezolizumab, nivolumab, and durvalumab.

Additional treatment modalities that may be used in combination with an atropisomer described herein, or a salt thereof include radiotherapy, a monoclonal antibody against a tumor antigen, a complex of a monoclonal antibody and toxin, a T-cell adjuvant, bone marrow transplant, or antigen presenting cells (e.g., dendritic cell therapy).

The present disclosure contemplates the use of atropisomers described herein, or a salt thereof for the treatment of glioblastoma either alone or in combination with radiation and/or temozolomide (TMZ), avastin or lomustine.

The present disclosure encompasses pharmaceutically acceptable salts, acids or derivatives of any of the above.

### Dosing

The atropisomers described herein, or a salt thereof may be administered to a subject in an amount that is dependent upon, for example, the goal of administration (e.g., the degree of resolution desired); the age, weight, sex, and health and physical condition of the subject to which the formulation is being administered; the route of administration; and the nature of the disease, disorder, condition or symptom thereof. The dosing regimen may also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

In general, dosing parameters dictate that the dosage amount be less than an amount that could be irreversibly toxic to the subject (the maximum tolerated dose (MTD)) and not less than an amount required to produce a measurable effect on the subject. Such amounts are determined by, for example, the pharmacokinetic and pharmacodynamic parameters associated with ADME, taking into consideration the route of administration and other factors.

An effective dose (ED) is the dose or amount of an agent that produces a therapeutic response or desired effect in some fraction of the subjects taking it. The “median effective dose” or ED₅₀ of an agent is the dose or amount of an agent that produces a therapeutic response or desired effect in 50% of the population to which it is administered. Although the ED₅₀ is commonly used as a measure of reasonable expectance of an agent's effect, it is not necessarily the dose that a clinician might deem appropriate taking into consideration all relevant factors. Thus, in some situations the effective amount is more than the calculated ED₅₀, in other situations the effective amount is less than the calculated ED₅₀, and in still other situations the effective amount is the same as the calculated ED₅₀.

In addition, an effective dose of atropisomers described herein, or a salt thereof may be an amount that, when administered in one or more doses to a subject, produces a desired result relative to a healthy subject. For example, for a subject experiencing a particular disorder, an effective dose may be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

In certain embodiments, the atropisomers described herein, or a salt thereof may be administered (e.g., orally) at dosage levels of about 0.01 mg/kg to about 50 mg/kg, or about 1 mg/kg to about 25 mg/kg, of subject body weight per day, one or more times a day, to obtain the desired therapeutic effect.

For administration of an oral agent, the compositions can be provided in the form of tablets, capsules and the like containing from 1.0 to 1000 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient.

In certain embodiments, the dosage of an atropisomer described herein, or a salt thereof is contained in a “unit dosage form”. The phrase “unit dosage form” refers to physically discrete units, each unit containing a predetermined amount an atropisomer or a subembodiment described herein, or a salt thereof, either alone or in combination with one or more additional agents, sufficient to produce the desired effect. It will be appreciated that the parameters of a unit dosage form will depend on the particular agent and the effect to be achieved.

### Kits

The present invention also contemplates kits comprising an atropisomer described herein, or a salt thereof, and pharmaceutical compositions thereof. The kits are generally in the form of a physical structure housing various components, as described below, and may be utilized, for example, in practicing the methods described above.

A kit can include one or more of an atropisomer described herein, or a salt thereof (provided in, e.g., a sterile container), which may be in the form of a pharmaceutical composition suitable for administration to a subject. The atropisomers described herein, or a salt thereof can be provided in a form that is ready for use (e.g., a tablet or capsule) or in a form requiring, for example, reconstitution or dilution (e.g., a powder) prior to administration. When the atropisomers described herein, or a salt thereof are in a form that needs to be reconstituted or diluted by a user, the kit may also include diluents (e.g., sterile water), buffers, pharmaceutically acceptable excipients, and the like, packaged with or separately from the atropisomers described herein, for a salt thereof. When combination therapy is contemplated, the kit may contain the several agents separately or they may already be combined in the kit. Each component of the kit may be enclosed within an individual container, and all of the various containers may be within a single package. A kit of the present invention may be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### Exemplary Embodiments

Embodiment 1: A compound having Formula (Ia)

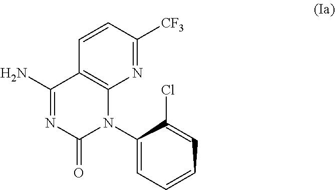

or a pharmaceutically acceptable salt thereof.

Embodiment 2: A composition comprising an atropisomer of Formula (Ia)

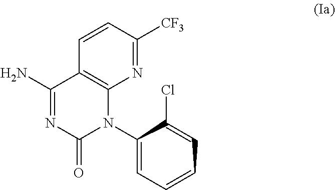

or a pharmaceutically acceptable salt thereof wherein the enantiomeric ratio of atropisomers is at least 60:40 of the displayed atropisomer.

Embodiment 3: The composition of embodiment 2, wherein the enantiomeric ratio of atropisomers is at least 65:35 of the displayed atropisomer.

Embodiment 4: The composition of embodiment 2, wherein the enantiomeric ratio of atropisomers is at least 70:30 of the displayed atropisomer.

Embodiment 5: The composition of embodiment 2, wherein the enantiomeric ratio of atropisomers is at least 75:25 of the displayed atropisomer.

Embodiment 6: The composition of embodiment 2, wherein the enantiomeric ratio of atropisomers is at least 80:20 of the displayed atropisomer.

Embodiment 7: The composition of embodiment 2, wherein the enantiomeric ratio of atropisomers is at least 85:15 of the displayed atropisomer.

Embodiment 8: The composition of embodiment 2, wherein the enantiomeric ratio of atropisomers is at least 90:10 of the displayed atropisomer.

Embodiment 9: The composition of embodiment 2, wherein the enantiomeric ratio of atropisomers is at least 95:5 of the displayed atropisomer.

Embodiment 10: The composition of embodiment 2, wherein the enantiomeric ratio of atropisomers is at least 99:1 of the displayed atropisomer.

Embodiment 11: A pharmaceutical composition comprising the compound of embodiment 1 or the composition of any one of embodiments 2 to 10, and at least one pharmaceutically acceptable excipient.

Embodiment 12: A method for treating a disease treatable by inhibition of MAT2A in a patient comprising administering to the patient a therapeutically effective amount of the compound of embodiment 1, the composition of any one of embodiments 2 to 10, or the pharmaceutical composition of embodiment 11.

Embodiment 13: The method of embodiment 12, wherein the disease is cancer.

Embodiment 14: A method of treating a MTAP null cancer in a patient comprising administering to the patient a therapeutically effective amount of the compound of embodiment 1, the composition of any one of embodiment 2 to 10, or the pharmaceutical composition of embodiment 11.

Embodiment 15: A method for treating a cancer in a patient, wherein the cancer is characterized by a reduction or absence of MTAP gene expression, the absence of the MTAP gene, reduced level of MTAP protein, or reduced function of MTAP protein, comprising administering to the subject a therapeutically effective amount of the compound of embodiment 1, the composition of any one of embodiments 2 to 10, or the pharmaceutical composition of embodiment 11.

Embodiment 16: The method of any one of embodiments 12 to 15, wherein the cancer is selected from the group consisting of leukemia, esophageal cancer, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, soft tissue sarcoma, non-Hodgkin lymphoma and mesothelioma.

Embodiment 17: A method of treating cancer in a patient comprising administering to the patient a therapeutically effective amount of the compound of embodiment 1, the composition of any one of embodiments 2 to 10, or the pharmaceutical composition of embodiment 11.

### Additional Exemplary Embodiments

Embodiment A-1: A compound having Formula (Ia)

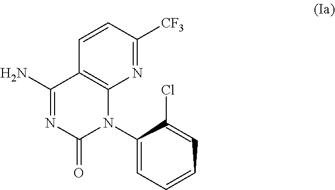

or a pharmaceutically acceptable salt thereof.

Embodiment A-2: The compound of Embodiment A-1, wherein the compound is Formula (Ia)

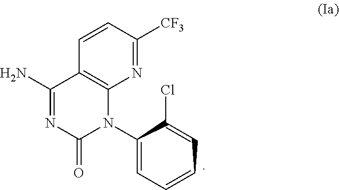

Embodiment A-3: A composition comprising an atropisomer of Formula (Ia)

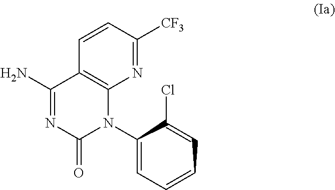

or a pharmaceutically acceptable salt thereof, wherein the enantiomeric ratio of atropisomers is at least 60:40 of the displayed atropisomer, and at least one or more pharmaceutically acceptable excipient.

Embodiment A-4: The composition of Embodiment A-3, wherein the enantiomeric ratio of atropisomers is at least 65:35 of the displayed atropisomer.

Embodiment A-5: The composition of Embodiment A-3, wherein the enantiomeric ratio of atropisomers is at least 70:30 of the displayed atropisomer.

Embodiment A-6: The composition of Embodiment A-3, wherein the enantiomeric ratio of atropisomers is at least 75:25 of the displayed atropisomer.

Embodiment A-7: The composition of Embodiment A-3, wherein the enantiomeric ratio of atropisomers is at least 80:20 of the displayed atropisomer.

Embodiment A-8: The composition of Embodiment A-3, wherein the enantiomeric ratio of atropisomers is at least 85:15 of the displayed atropisomer.

Embodiment A-9: The composition of Embodiment A-3, wherein the enantiomeric ratio of atropisomers is at least 90:10 of the displayed atropisomer.

Embodiment A-10: The composition of Embodiment A-3, wherein the enantiomeric ratio of atropisomers is at least 95:5 of the displayed atropisomer.

Embodiment A-11: The composition of Embodiment A-3, wherein the enantiomeric ratio of atropisomers is at least 99:1 of the displayed atropisomer.

Embodiment A-12: A pharmaceutical composition comprising the compound of Embodiment A-1 or A-2 or the composition of any one of Embodiments A-3 to A-11, and at least one pharmaceutically acceptable excipient.

Embodiment A-13: A method for treating a disease treatable by inhibition of MAT2A in a patient comprising administering to the patient a therapeutically effective amount of the compound of Embodiment A-1 or A-2, the composition of any one of Embodiments A-3 to A-11, or the pharmaceutical composition of Embodiment A-12.

Embodiment A-14: The method of Embodiment A-13, wherein the patient is in recognized need of such treatment.

Embodiment A-15: The method of Embodiment A-13 or A-14, wherein the disease is cancer.

Embodiment A-16: A method of treating cancer in a patient comprising administering to the patient a therapeutically effective amount of the compound of Embodiment A-1 or A-2, the composition of any one of Embodiments A-3 to A-11, or the pharmaceutical composition of Embodiment A-12.

Embodiment A-17: The method of Embodiment A-16, wherein the patient is in recognized need of such treatment.

Embodiment A-18: A method of treating a MTAP null cancer in a patient comprising administering to the patient a therapeutically effective amount of the compound of Embodiment A-1 or A-2, the composition of any one of Embodiments A-3 to A-11, or the pharmaceutical composition of Embodiment A-12.

Embodiment A-19: The method of Embodiment A-18, wherein the patient is in recognized need of such treatment.

Embodiment A-20: A method for treating a cancer in a patient, wherein the cancer is characterized by a reduction or absence of MTAP gene expression, the absence of the MTAP gene, reduced level of MTAP protein, reduced function of MTAP protein, MTA accumulation, or combination thereof, comprising administering to the subject a therapeutically effective amount of the compound of Embodiment A-1 or A-2, the composition of any one of Embodiment A-3 to A-11, or the pharmaceutical composition of Embodiment A-12.

Embodiment A-21: The method of Embodiment A-20, wherein the patient is in recognized need of such treatment.

Embodiment A-22: The method of any one of Embodiment A-15 to A-21, wherein the cancer is selected from the group consisting of leukemia, esophageal cancer, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, soft tissue sarcoma, non-Hodgkin lymphoma and mesothelioma.

Embodiment A-23: The method of any one of Embodiments A-15 to A-21, wherein the cancer is selected from the group consisting of non-small cell lung cancer (squamous and adenocarcinoma), urothelial cancer (bladder and upper urinary tract), esophageal cancer, and gastric cancer.

### EXAMPLES

The following examples and intermediates are put forth so as to provide those of ordinary skill in the art with a complete disclosure and description of how to make and use the present invention, and are not intended to limit the scope of what the inventors regard as their invention. It is to be understood that exemplary descriptions written in the present tense were not necessarily performed, but rather that the descriptions can be performed to generate data and the like of a nature described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used.

### Example 1: Preparation of a Compound of Formula I and Separation of Atropisomers Formula Ia and Ib

Formula I was prepared using the methods described in WO2020/123395.

Subsequent chromatography experiments identified that Formula I could be separated into atropisomers: Formula Ia and Formula Ib. Two chomatrography methods were used to separate Formula Ia and Formula Ib:

### Method A: (SFC)

Instrument: Waters UPC2 analytical SFC (SFC-H)
Column: ChiralCel OX, 100×4.6 mm I.D., 3 μm
Mobile phase: A for CO₂ and B for Methanol (0.05% DEA)
Gradient: B 40%
Flow rate: 2.5 mL/min
Back pressure: 100 bar
Column temperature: 35° C.
Wavelength: 220 nm

### Method B: (Preparative)

Instrument: Shimadzu LC-20AP HPLC
Column: ChiralCel OX, 300×50 mm I.D., 10 μm
Mobile phase: Ethanol
Flow rate: 120 mL/min
Column temperature: R.T.
Wavelength: 220 nm

Prior to chromatography, Formula I (150 g) was dissolved in 12 L ethanol/DCM (9:1). Eluted fractions from the column were dried off via rotary evaporator. After concentration the enantiomeric excess (e.e.) was tested via SFC.

Retention time in Method A was about 1.36 min for Formula Ib and about 3.45 min for Formula Ia. As determined by SFC, the e.e. for isolated Formula Ib was determined to be about 97%, and the e.e, for isolated Formula Ia was determined to be 96%. Absolute stereochemistry of Formula Ia (2^{nd} eluting peak) was determined by a co-crystal structure in the MAT2A enzyme.

The specific rotation observed for Formula Ia and Formula Ib are reported in Table 1.

TABLE 1

| Atropisomer | Specific Optical Rotation $( {⌈ α ⌉}_{D}^{25} )$ |
|---|---|
| Ia | +23.6 |
| (C = 0.996 g/100 mL | |
| in Methanol) | |
| Ib | −23.1 |
| (C = 0.974 g/100 mL | |
| in methanol) | |

### Example 2: Atropisomer of Formula Ia Provides a Surprising Improvement in Activity Over Atropisomer of Formula Ib

The following example compares the activity of Atropisomers of Formula Ia and Ib in: (A) an in vitro fluorescence assay, and (B) a cell viability assay.

### A: Phosphate Sensor Fluorescence Assay

MAT2A enzyme is incubated with a test compound in DMSO or DMSO and its substrates (L-methionine and ATP) in a microtiter plate. The enzymatic reaction is stopped by the addition of Working Phosphate Sensor Mixture. The plate is analyzed for fluorescence at 450 nm. The high control (DMSO with enzyme and its substrates) gives high fluorescence which represents no inhibition of enzymatic activity while the low control (DMSO with MAT2A substrates and no enzyme) gives low fluorescence which represents full inhibition of enzymatic activity.

### Materials

Human MAT2A: Cepter, amino acids 1-395 Clone ID 334, lot 00023-148
Tris, pH 7.5: Invitrogen cat #15567-027
KCl: Ambion cat #AM9640G
MgCl₂: Ambion cat #AM9530G
Brij-35: Sigma cat B4184-10ML
DTT: Goldbio cat #DTT100
BGG: Sigma cat #G5009-25G
PNP: Novus Biologicals cat #NBP1-50872
7-MEG: Cayman Chemical cat #15988
L-Methionine: JTBaker P/N 2085-05
ATP: Promega P/N V915B
Phosphate Sensor: Invitrogen cat #PV4407
EDTA: Life Tech cat #15575-038
Assay plate: 384-well black polypropylene plate: Corning P/N 3573

### Final Assay Conditions:

Assay Buffer: 50 mM Tris, pH 7.5/50 mM KCl/10 mM MgCl₂/0.01% Brij-35/1 mM DTT/0.01% BGG/40 nM PNP/6 uM 7-MEG

MAT2A: 5.8 nM MAT2A (1-395), Cepter, clone ID 334, lot 00023-148 before the addition of Working Phosphate Sensor Mixture

L-methionine: 500 uM before the addition of Working Phosphate Sensor Mixture

ATP: 500 uM before the addition of Working Phosphate Sensor Mixture

### Procedure:

For the assay, a mixture of 1 mM L-methionine/1 mM ATP (2× final pre-stopped concentration) in assay buffer: MAT2A (2× final pre-stopped concentration) in Assay Buffer and Working Phosphate Sensor Mixture (1.5 uM Phosphate Sensor/30 mM EDTA in Assay Buffer, which is 3× final concentrations) were prepared. Test compounds or DMSO were added to the appropriate well using a Beckman Coulter Echo 550 acoustic liquid handler. 10 μL/well of Assay Buffer was added to the wells corresponding to the negative control and 10 μL/well of MAT2A was added to all the wells except for those corresponding to the negative control. After incubating the plate at room temperature for 15 minutes, 10 μL/well of the 1 mM L-methionine/1 mM ATP mixture was added to all wells. The plate was centrifuged at 1000 rpm for 1 minute and then incubated at room temperature for 1 hour. 10 μL of the Working Phosphate Sensor Mixture was added to all wells and the plate was centrifuged at 1000 rpm for 1 minute. The plate was read for fluorescence at 450 nm after exciting at 430 nm on a Tecan Spark microplate reader.

### Data Analysis:

Percent inhibition was calculated in Chemical and Biological Information System (CBIS), (ChemInnovation Software Inc.). Curves were fitted by CBIS as % inhibition vs. log [compound concentration] using a 4-parameter inhibition model.

$$Fit = ( A + ( ( B - A ) / ( 1 + ( ( C / x ) \hat{} D ) ) ) )$$

$$Res = ( y - fit )$$

The IC₅₀ of each atropisomer is shown in Table 2, below.

TABLE 2

| Atropisomer | IC₅₀ |
|---|---|
| Ia | 3 nM |
| Ib | 60 nM |

### B: HCT116 MTAP-Null Cell Line Viability

### Prepare Cells for Assay:

Preparation: HCT116 WT and MTAP null cells were cultured in DMEM/F12+10% FBS at 37° C. Left the media, dissociation reagent, and PBS in room temperature at least 1 hr prior to start.

Process: Washed cells with 5 mL of PBS, aspirate the PBS. Added 1 mL of dissociation reagent (Trypsin, Gibco #25200114) and left at room temperature for 2-5 mins. When cells were detached, added 5 ml of fresh media. Cells were collected into a 10 mL tube and centrifuged at 1000 rpm for 5 minutes at room temperature. Aspirated media, resuspended in 5 mL fresh media, and counted on Bio-Rad TC20 Cell Counter. Based on the count and the table below, added cells to a clear flat bottom, TC-treated 384-well plate (Corning #3764).

| cell | | | vol | Total | Total |
|---|---|---|---|---|---|
| line | cells | wells | (ml) | cells | vol(ml) |
| HCT116 | 300 | 768 | 0.045 | 2.304E6 | 34.6 |

Treatment with the Compounds:

Treated the cells the next day post splitting. Used a 10 mM stock of compound.

Process: Compound treatment was applied to cells using a Tecan D300e digital dispenser with a high concentration 20 micromolar and 3-fold serial dilutions over 9 concentrations in triplicate on a 384-well plate.

### Final Count:

4-5 days post treatment, used a Live-Cell Analysis System (IncuCyte®) suitable for reading GFP to do a final count. Raw data were normalized based on the average value of the DMSO wells on each plate.

The EC₅₀ of each atropisomer is shown in Table 3, below.

TABLE 3

| Atropisomer | EC50 |
|---|---|
| Ia | 20 nM |
| Ib | 149 nM |

What is claimed:

1. A compound having Formula (Ia) 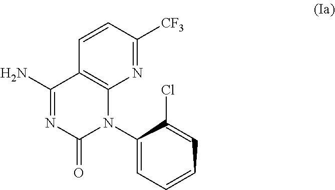
or a pharmaceutically acceptable salt thereof.

2. The compound of claim 1, wherein the compound is Formula (Ia)

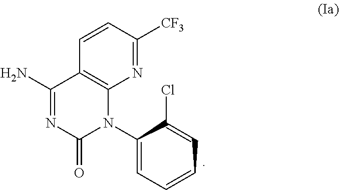

3. A composition comprising an atropisomer of Formula (Ia) 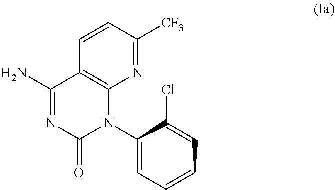
or a pharmaceutically acceptable salt thereof, wherein the enantiomeric ratio of atropisomers is at least 60:40 of the displayed atropisomer.

4. The composition of claim 3, wherein the enantiomeric ratio of atropisomers is at least 65:35 of the displayed atropisomer.

5. The composition of claim 3, wherein the enantiomeric ratio of atropisomers is at least 70:30 of the displayed atropisomer.

6. The composition of claim 3, wherein the enantiomeric ratio of atropisomers is at least 75:25 of the displayed atropisomer.

7. The composition of claim 3, wherein the enantiomeric ratio of atropisomers is at least 80:20 of the displayed atropisomer.

8. The composition of claim 3, wherein the enantiomeric ratio of atropisomers is at least 85:15 of the displayed atropisomer.

9. The composition of claim 3, wherein the enantiomeric ratio of atropisomers is at least 90:10 of the displayed atropisomer.

10. The composition of claim 3, wherein the enantiomeric ratio of atropisomers is at least 95:5 of the displayed atropisomer.

11. The composition of claim 3, wherein the enantiomeric ratio of atropisomers is at least 99:1 of the displayed atropisomer.

12. A pharmaceutical composition comprising the compound of claim 1 or claim 2, or the composition of any one of claims 3 to 11, and at least one pharmaceutically acceptable excipient.

13. A method for treating a disease treatable by inhibition of MAT2A in a patient comprising administering to the patient a therapeutically effective amount of the compound of claim 1 or claim 2, the composition of any one of claims 3 to 11, or the pharmaceutical composition of claim 12.

14. The method of claim 13, wherein the patient is in recognized need of such treatment.

15. The method of claim 13 or 14, wherein the disease is cancer.

16. A method of treating a MTAP null cancer in a patient comprising administering to the patient a therapeutically effective amount of the compound of claim 1 or claim 2, the composition of any one of claims 3 to 11, or the pharmaceutical composition of claim 12.

17. The method of claim 16, wherein the patient is in recognized need of such treatment.

18. A method for treating a cancer in a patient, wherein the cancer is characterized by a reduction or absence of MTAP gene expression, the absence of the MTAP gene, reduced level of MTAP protein, or reduced function of MTAP protein, comprising administering to the subject a therapeutically effective amount of the compound of claim 1 or claim 2, the composition of any one of claims 3 to 11, or the pharmaceutical composition of claim 12.

19. The method of claim 18, wherein the patient is in recognized need of such treatment.

20. The method of any one of claims 15 to 19, wherein the cancer is selected from the group consisting of leukemia, esophageal cancer, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, soft tissue sarcoma, non-Hodgkin lymphoma and mesothelioma.

21. The method of any one of claims 15 to 19, wherein the cancer is selected from the group consisting of non-small cell lung cancer (squamous and adenocarcinoma), urothelial cancer (bladder and upper urinary tract), esophageal cancer, and gastric cancer.

22. A method of treating cancer in a patient comprising administering to the patient a therapeutically effective amount of the compound of claim 1 or claim 2, the composition of any one of claims 3 to 11, or the pharmaceutical composition of claim 12.
